(19) United States
(12) Patent Application Publication
(10) Pub. No.: US 2011/0144514 A1
(43) Pub. Date: Jun. 16, 2011

(54) Tracheal Tube with Pressure Monitoring Lumen and Method for Using the Same

(75) Inventor: Jeffrey A. Booker, Broomfield, CO (US)

(73) Assignee: NELLCOR PURITAN BENNETT LLC, BOULDER, CO (US)

(21) Appl. No.: 12/639,588

(22) Filed: Dec. 16, 2009

(51) Int. Cl. A61B 5/08; A61M 16/04

(52) U.S. Cl. 600/529; 128/207.14

(57) ABSTRACT

According to various embodiments, methods and systems for determining pressure in the lungs may employ tracheal pressure measurements. The tracheal pressure measurements may be obtained through a pressure monitoring lumen associated with a tracheal tube. Such systems may include a purging or flushing mechanism to keep the pressure monitoring lumen free of any obstructions. The flushing mechanism may utilize respiratory gases diverted from the airway stream and regulated to flush the lumen at relatively low pressures. The resulting pressure measurements may be used to determine a more accurate estimate of lung pressure, which in turn may be used to control a ventilator and provide breathing assistance to a patient.

### BACKGROUND

The present disclosure relates generally to medical devices and, more particularly, to airway devices, such as tracheal tubes.

This section is intended to introduce the reader to aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air, food, fluids, or other substances into the patient. For example, tracheal tubes may be used to control the flow of air or other gases through a patient's trachea and into the lungs, for example during patient ventilation. Such tracheal tubes may include endotracheal (ET) tubes, tracheotomy tubes, or transtracheal tubes. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

To seal these types of tracheal tubes, an inflatable cuff may be associated with the tubes. When inflated, the cuff generally expands into the surrounding trachea to seal the tracheal passage around the tube to facilitate the controlled delivery of gases via a medical device (e.g., through the tube). For intubated patients, the flow rate and volume of gas transferred into the lungs, which may vary according to the condition of each patient, may be controlled by the settings of a ventilator. One factor that is used to determine the ventilator settings may be an airway pressure measurement, which is typically obtained by measuring the pressure along the breathing circuit (e.g., medical tubing connecting the tracheal tube to the ventilator) at a point outside the patient. Airway pressure measured in the breathing circuit at a point outside the patient may be a useful surrogate for the pressure in the lungs, which may in turn be used for calculating a number of ventilator settings, for example settings involving pressure limits.

However, in circumstances where the internal diameter of the tracheal tube is diminished, for example through the buildup of mucosal secretions that may partially block the airflow passage of the tracheal tube, the lung pressure may be different from the airway pressure measurement taken outside the patient. Accordingly, an airway pressure measurement may not always serve as a reliable substitute for lung pressure measurements.

### BRIEF DESCRIPTION OF THE DRAWINGS

Advantages of the disclosure may become apparent upon reading the following detailed description and upon reference to the drawings in which:

FIG. 1 illustrates a system including an endotracheal tube with a pressure monitoring lumen according to embodiments of the present techniques;

FIG. 2 is a block diagram of an example of a pressure monitoring lumen purging system that may be used in conjunction with the system of FIG. 1;

FIG. 3 is a perspective view of an endotracheal tube with a pressure monitoring lumen that may be used in conjunction with the system of FIG. 1;

FIG. 4 is a flow diagram of an exemplary method for deriving trachea pressure; and

FIG. 5 is a plot of exemplary respiratory pressure.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Because direct measurements of the pressure in the internal space of the lungs is difficult, clinicians and respiratory specialists may use surrogate measurements of pressure along various points of breathing circuit or the patient's airway to estimate the lung pressure. The lung pressure estimates may then be used to determine the efficacy of the ventilation (e.g., the dynamic intrapulmonary compliance) and, in some cases, may be used to control the settings of a ventilator, either manually or automatically, to provide a clinical benefit to the patient.

Airway pressure may be estimated by using measurements of pressure taken along various points of the breathing circuit that are proximal to the tracheal tube. For example, such measurements may be used to assess a patient's work of breathing, which may include the airway resistance during movement of air into and out of the lungs. If the work of breathing of the patient increases, clinicians may assess whether the increase is due to increased airway resistance in the patient (e.g., stiffened lung tissue, which may be related to a clinical condition) or increased resistance in the tracheal tube due to buildup of biofilms on the inner diameter of the tube. Because airway pressure measurements taken proximal to the tracheal tube may not provide information about resistance built up distally, either in the patient or in the tube, trachea pressure measurements may provide information to the clinician about airway or tube-originated resistance. Trachea pressure may refer to pressure in the airway space below the cuff and/or near the distal tip of the tracheal tube.

In particular, because the internal diameter of tracheal tube may change during the time that the patient is intubated (e.g., a buildup of patient secretions within the tube may change the inner diameter), measurements taken upstream of the tracheal tube in the breathing circuit may not be reliable for estimating pressure in the lungs. In certain embodiments, a measurement of tracheal pressure may be used as a surrogate for lung pressure or other pulmonary pressure measurements. The tracheal space is contiguous with the lung space, and tracheal pressure may be a more reliable measurement than measurements taken far upstream along the breathing circuit. Trachea pressure may be determined by using pressure transducers inserted at the distal end of the endotracheal tube or by sampling the gas in the tracheal space with a lumen connected to a proximally located pressure transducer. However, during long-term patient monitoring, the distal end of the tracheal tube may become covered in mucus or secretions, which may interfere with a pressure transducer located at the distal end of the tube or which may block a pressure monitoring lumen. For example, when a patient coughs, mucus from the lungs may be deposited at the distal end of the tracheal tube. When the pressure transducer or pressure monitoring lumen is covered in mucus, measurement accuracy may be affected.

Accordingly, the disclosed embodiments provide a more accurate method and system for determining trachea pressure by providing a tracheal tube with a pressure monitoring lumen that samples gas at or near the distal end of the tracheal tube. The pressure monitoring lumen may be kept clear of mucus blockage through flushing of the lumen with a fluid, such as a gas. By diverting a small portion of respiratory source gas for use in flushing the pressure monitoring lumen, the lumen may be kept free of obstructions. The pressure of the flushing gas through the lumen may be carefully controlled so that the additional gas provided through the pressure monitoring lumen has little effect on the total pressure in the lungs. In addition, by keeping a substantially constant pressure and flow rate through the pressure monitoring lumen, the effect of the flushing gas on pressure measurements may be compensated for or, in embodiments in which the flushing pressure is relatively low, ignored.

In certain presently contemplated embodiments, the trachea pressure may be used to evaluate, adjust, or correct airway pressure values obtained along the breathing circuit or ventilator settings. For example, if the estimate of trachea pressure varies significantly from the airway pressure measured upstream at a point closer to the ventilator, a clinician may be able to determine that the tracheal tube is blocked with secretions or other buildup, or that some other condition has developed, which may involve action by the clinician.

In certain embodiments, the disclosed tracheal tubes, systems, and methods may be used in conjunction with any appropriate medical device, including a feeding tube, an endotracheal tube, a tracheotomy tube, a circuit, an airway accessory, a connector, an adapter, a filter, a humidifier, a nebulizer, nasal cannula, or a supraglottal mask/tube. The present techniques may also be used to monitor any patient benefiting from mechanical ventilation, e.g., positive pressure ventilation. Further, the devices and techniques provided herein may be used to monitor a human patient, such as a trauma victim, an intubated patient, a patient with a tracheotomy, an anesthetized patient, a cardiac arrest victim, a patient suffering from airway obstruction, or a patient suffering from respiratory failure.

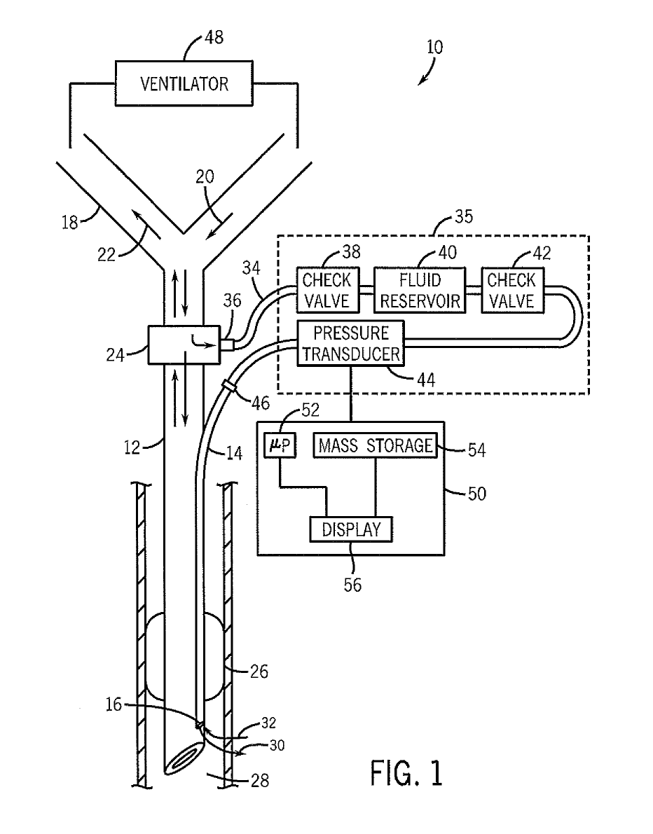

FIG. 1 shows an exemplary tracheal tube system 10 that has been inserted into the trachea of a patient. The system 10 includes a tracheal tube 12, shown here as an endotracheal tube, with a pressure monitoring lumen 14 that may be incorporated into the walls of the tracheal tube, e.g., the lumen 14 may be coextruded in the walls. The pressure monitoring lumen 14 may terminate in an opening 16 formed in the walls of the tracheal tube 12 to allow the pressure monitoring lumen to be in fluid communication with the patient airway.

The system 10 may also include a respiratory circuit connected to the endotracheal tube 12 that allows one-way flow of expired gases away from the patient and one-way flow of inspired gases towards the patient. For example, the system 10 may include a Y-connector 18 in fluid communication with a source of respiratory gas. The Y-connector 18 may include a branch for airflow flow into the lungs (i.e., inspiration), represented by arrow 20, and airflow out of the lungs (i.e., exhalation), represented by arrow 22. The system 10 may include any number of other connectors or medical tubing to provide respiratory gases from a gas source to the lungs. For example, connector 24 may couple the Y-connector 18 to the proximal end of the tracheal tube 12. The respiratory circuit, including the tube 12, may include standard medical tubing made from suitable materials such as polyurethane, polyvinyl chloride (PVC), polyethylene teraphthalate (PETP), low-density polyethylene (LDPE), polypropylene, silicone, neoprene, polytetrafluoroethylene (PTFE), or polyisoprene.

The tracheal tube 12 may also be associated with an inflatable cuff 26 that functions to form a seal against the tracheal walls and isolate the lower airway space 28 of the lower trachea and lungs during mechanical ventilation. The pressure monitoring lumen 14 is configured to sample air from the lower airway space 28. The system 10 also includes a mechanism for keeping flow through the pressure monitoring lumen 14 so that blockages do not form around the opening 16. As such, the pressure monitoring lumen 14 has continuous, or in certain embodiments, sporadic airflow out of the opening 16, represented by arrow 30, and may also receive airflow into the lumen, represented by arrow 32. Given that the airflow to the lumen is bidirectional, the pressure in the lumen 14 may represent an equilibrated pressure from the inflow and outflow components.

The pressure monitoring lumen 14 is in fluid communication with a purging system 35 that diverts respiratory gases from a point on the airway circuit. As shown, the connection point 36 may be on coupler 24 or on any other suitable location. The diverted gases may flow into a conduit 34 into a one-way check valve 38 that helps trap the gases in fluid reservoir 40. Fluid reservoir 40 may be configured to accumulate gases and then allow the gases to be released at a known or controlled rate to purge the pressure monitoring lumen 14. Another check valve 42 may be on the opposing side of the fluid reservoir 40 to prevent air coming from the pressure monitoring lumen 14 from accumulating in the fluid reservoir 40. A pressure transducer 44 is on the tracheal tube side of the fluid reservoir 40 and is positioned to be in fluid communication with the conduit 34 and the pressure monitoring lumen 14. It should be understood that conduit 34 may include any number of additional conduits and couplers in order to couple the various elements of the purging system. As shown, the pressure transducer 44 may be on or within conduit 34, which in turn may be in fluid communication with pressure monitoring lumen 14. In other embodiments, the pressure transducer 44 may be a part of coupler 46, which may connect conduit 34 and purging system 35 to pressure monitoring lumen 14. In other embodiments, the pressure transducer 44 may be part of a proximal portion of pressure monitoring lumen 14 and may not be part of the purging system 35.

The system 10 may also include devices that facilitate positive pressure ventilation of a patient, such as the ventilator 48, which may include any ventilator, such as those available from Nellcor Puritan Bennett LLC. The system may also include a monitor 50 that may be configured to implement embodiments of the present disclosure to determine pressures based upon the pressure detected by the pressure transducer 44. It should be understood that the monitor 50 may be a stand-alone device or may, in embodiments, be integrated into a single device with, for example, the ventilator 48.

The monitor 50 may include processing circuitry, such as a microprocessor 52 coupled to an internal bus and a display 56. In certain embodiments, the system 10 may also provide calibration information for the purging mechanism and/or pressure transducer 44. The information may then be stored in mass storage device 54, such as RAM, PROM, optical storage devices, flash memory devices, hardware storage devices, magnetic storage devices, or any suitable computer-readable storage medium. The information may be accessed and operated upon according to microprocessor 52 instructions. In certain embodiments, the information may be used in calculations for estimating of pressure in the lungs. The monitor 50 may be configured to provide indications of the lung pressure, such as an audio, visual or other indication, or may be configured to communicate the estimated lung pressure to another device, such as the ventilator 48.

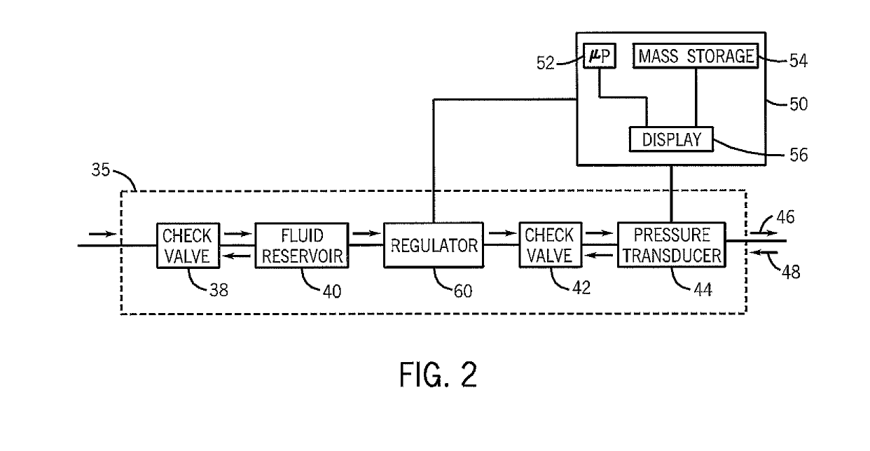

FIG. 2 is block diagram of certain components of the purging system 35. Diverted respiratory gases from the airway circuit may enter conduit 34. Check valve or one-way valve 38, in-line with conduit 34, prevents gases, once they have passed the point of the valve 38, from being transferred back through conduit 34 and connection point 36 into the respiratory stream. This allows gases to flow into and accumulate within the fluid reservoir 40 to a desired pressure. The fluid reservoir may be a tank or other accumulating structure in fluid communication with conduit 34 of a suitable size and shape to allow sufficient volume of accumulated gas to purge the pressure monitoring lumen 14. In certain embodiments, the fluid reservoir and/or the purging system 35 may include a micropump to facilitate the transfer of respiratory gases into the reservoir 40, while in other embodiments the purging system 35 does not include or require a pump. The inclusion of a pump may reduce or eliminate the need for check valves to prevent the undesired movement of gas back through connection point 36. The check valve 38 and additional valves (e.g., regulator 60) may be built into the structure of the fluid reservoir 40 or, in certain embodiments, may be separate structures.

In particular embodiments, the purging system 35 may also include a regulator to control the transfer of gases from the fluid reservoir. The regulator 60 may control the pressure from the relatively higher pressure environment of the fluid reservoir 40 to a lower pressure suitable for purging the pressure monitoring lumen 14. For example, the fluid reservoir may allow fluid to build up to pressure of about 30 cm H₂O. A regulator may reduce this higher pressure to a pressure of less than 10 cm H₂O. It is contemplated that the purging system 35 may be adapted to run in a substantially continuous manner during the course of patient intubation. To this end, the fluid reservoir 40 may be sized and shaped so that the inflow and accumulation of respiratory gases is sufficient to keep the outflow of gas to the pressure monitoring lumen 14 relatively constant and/or sufficient for purging. In certain presently contemplated embodiments, the pressure regulator may provide a relatively constant pressure outflow so as to ensure the flow of gas through the pressure monitoring lumen during at least some of the respiratory (or ventilation) cycle. That is, when the trachea pressure is lower, during expiration, the regulated pressure may cause gas to flow through the lumen to the trachea. Conversely, when the trachea pressure is higher, the flow through the lumen may be reduced (or temporarily stopped in case of pressure equilibrium).

It is contemplated that the purging system 35 may operate without external control. In such embodiments, the valves 38 and 42, as well as regulator 60, may be manually operated. For example, the regulator may include a pressure reading to allow such manual operation. In other embodiments, for example as shown in FIG. 2, the regulator may be controlled by a processor-based device, such as monitor 50. In such embodiments, monitor 50 may control the pressure outflow from the fluid reservoir 40 to any appropriate pressure based on either coded instructions or user input.

The purging system 35 may include an additional check valve 42 downstream of the fluid reservoir 40 and, in embodiments, downstream of the regulator 60. The additional check valve 42 may serve to keep backflow (e.g., represented by arrow 48) from the pressure monitoring lumen 14 from entering the fluid reservoir 40, which may prevent pressure fluctuations in the pressure monitoring lumen 14 being masked by backflow gases entering the fluid reservoir 40. A pressure transducer 44 may be located downstream of the fluid reservoir 40, regulator 60, and check valve 42. The pressure transducer 44 may be any suitable pressure sensor that may be integrated into or onto the conduit 34 or other lumens or connectors as provided. For example, the pressure transducer 44 may be a piezoelectric pressure sensor.

The purging system 35 may be provided as a kit or unit, which may also include a coupler 24 that configured to be part of the respiratory circuit. The coupler 24 may include a connection point 36 for attaching the purging system conduit 34 to the respiratory circuit and may include a downstream connection point for attaching the conduit 34 to the pressure monitoring lumen 14 of tracheal tube 12. As noted, one or more components of the purging system 35 may be combined into single structures. For example, in one embodiment, the purging system may include conduit 40 in-line with a single fluid reservoir 40 with a check valve built into the reservoir 40 at the upstream end (e.g., proximal to connection point 36) and a pressure regulator 60 or check valve 42 built into the reservoir 40 at the downstream end. As such, the purging system 35 may be relatively simple to connect and operate. The pressure transducer 44 may or may not be included in the purging system 35, and may be located at the proximal end of the pressure monitoring lumen 14 or within connection point 46 or another connecting conduit.

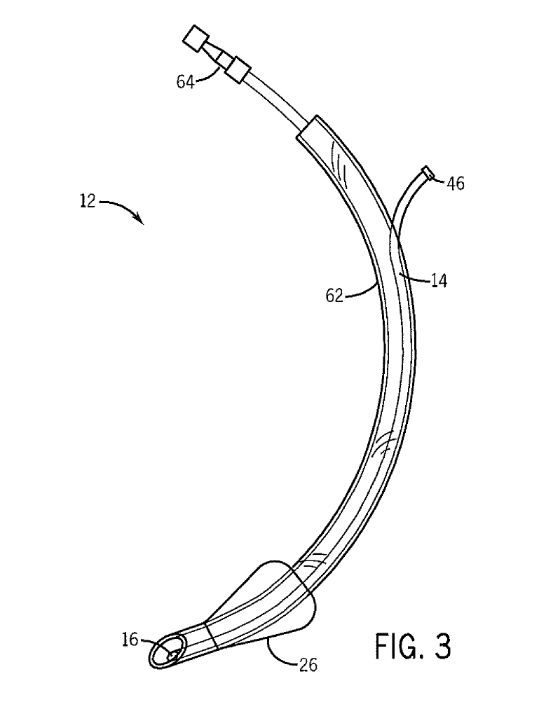

FIG. 3 is a perspective view of an exemplary tracheal tube 12 according to certain presently contemplated embodiments. The tracheal tube 12 includes a pressure monitoring lumen 14 that may be formed (e.g., through extrusion) in the tracheal walls 62. The lumen 14 terminates in an opening 16 that is distal to the cuff 26. As shown in FIG. 3, the opening 16 may be located on a slant portion of a distal end 64 of the tube 12. For example, the opening 16 may be formed by cutting a distal end of the tube and revealing the opening. In other embodiments, any opening at the slanted distal end 64 may be heat sealed and an opening 16 may be formed by puncturing or otherwise forming a hole in the tracheal walls 62 to access the lumen 14. The pressure monitoring lumen 14 may terminate in any suitable connector, such as connector 46, to facilitate fluid communication with the purging system 35.

The tube 12 may include a cuff 26 that may be inflated via a separate inflation lumen (not shown). In addition, the tube 12 may include a calibration element, such as connector 64, that may be suitably configured to connect to a receiving port on the monitor 50. The connector 64 may contain an information element, such as a memory circuit, such as an EPROM, EEPROM, coded resistor, or flash memory device for storing calibration information for the pressure monitoring lumen 14 and/or the purging system 35. It should be understood that the purging system 35 may also include such a connector 64 to facilitate calibration by the monitor 50. Alternatively, the pressure transducer 44 may include a passive or active RFID circuit that may be read wirelessly to convey pressure monitoring information and calibration information to the monitor 50. In other embodiments, tube identifying data, calibration data, and so forth may simply be entered manually.

The tube 12, the lumen 14, and the cuff 26 are formed from materials having suitable mechanical properties (such as puncture resistance, pin hole resistance, tensile strength), chemical properties (such as biocompatibility). In one embodiment, the walls of the cuff 26 are made of a polyurethane having suitable mechanical and chemical properties. An example of a suitable polyurethane is Dow Pellethane® 2363-80A. In another embodiment, the walls of the cuff 26 are made of a suitable polyvinyl chloride (PVC). In certain embodiments, the cuff 26 may be generally sized and shaped as a high volume, low pressure cuff that may be designed to be inflated to pressures between about 15 cm H₂O and 30 cm H₂O.

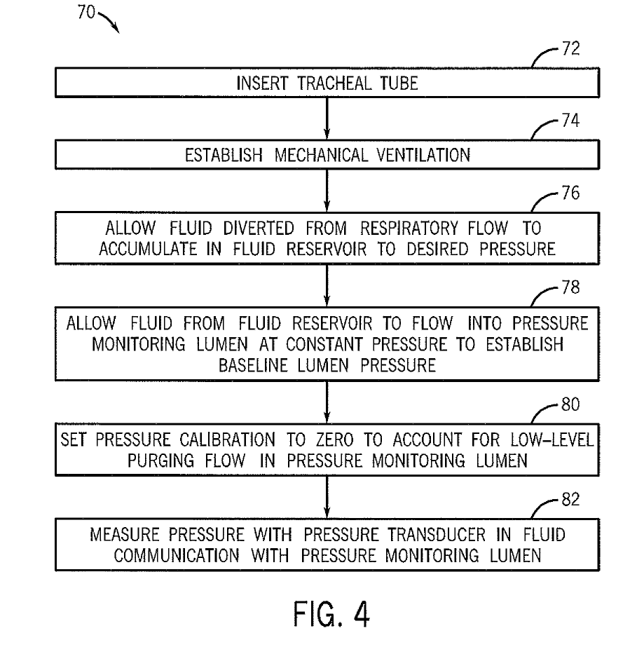

FIG. 4 is an exemplary process flow diagram illustrating a method for determining trachea pressure. The method is generally indicated by reference number 70 and includes various steps or actions represented by blocks. It should be noted that the method 70 may be performed as an automated or semiautomated procedure by a system, such as system 10. Further, certain steps or portions of the method may be performed by separate devices. For example, a portion of the method 70 may be performed by a pressure transducer 44, while another portion of the method 70 may be performed by a monitor 50. In embodiments, the method 70 may be performed continuously or intermittently for long-term patient monitoring or at any appropriate interval depending on the particular situation of the intubated patient.

According to a presently contemplated embodiment, the method 70 begins with the intubation of a patient at step 72. After the patient is intubated and the appropriate respiratory circuit components are put in place at step 74, including a purging system 35 in communication with tracheal tube 12, respiratory gases may be allowed to accumulate within fluid reservoir 40 to a desired pressure at step 76. The accumulated fluid, once established at the appropriate pressure within the reservoir 40, may then flow downstream within purging system 35 and into pressure monitoring lumen 14 at step 78.

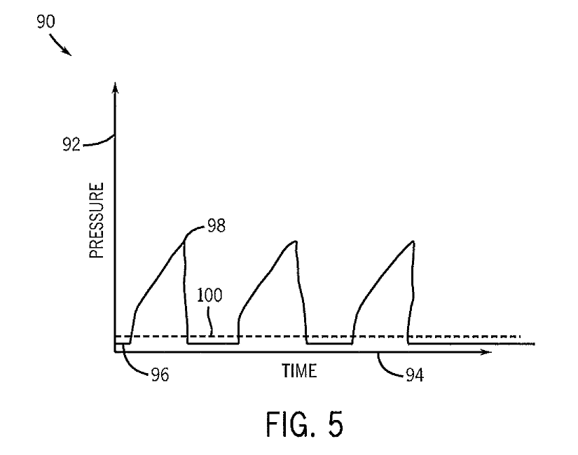

In certain embodiments, a baseline pressure in the lumen 14 may be established, and the pressure transducer 44 may be calibrated by setting the pressure at a particular point in the breathing cycle to a baseline, or zero, pressure at step 80 before measurement of pressure at step 82 takes place. For example, FIG. 5 is a plot 90 that shows an example of a patient respiratory cycle. The pressure delivered by the ventilator 48 is plotted on the y-axis 92 against time on the x-axis 94. Also shown is an example of a low-level constant purging pressure 100 applied to the pressure monitoring lumen 14. The purging pressure is greater than the minimum pressure 96, which represents the pressure left in the lungs after exhalation. The difference between the minimum pressure in the lungs 96 and the purging pressure 100 represents a potential source of calibration for the purging system 35. When purging pressure 100 is about or within 5% or 10% of the minimum pressure, the pressure may be simply ignored or factored out. In other embodiments, the monitor 50 may set a baseline pressure to the pressure 100 and determine any pressure changes based on increases from the baseline pressure. In addition, in certain embodiments, the purging pressure 100 is less than a peak inspiratory pressure 98. For ventilation settings configured to deliver pressure to the lungs of about 30 cm H₂O, the peak trachea pressure may be within 10% of 30 cm H₂O. In a presently contemplated embodiment, for specialized ventilation settings, such as PEEP, the purging pressure 100 may be adjusted accordingly, either manually or automatically.

The pressure measurements from the pressure transducer 44 may be communicated to the monitor 50 for further analysis. The monitor 50 may also receive calibration information from an information element or other storage device associated with the connector 64. It should be noted that the monitor may, of course, receive data or signals directly from the pressure transducer 44. Trachea pressure may be estimated from the pressure in the pressure monitoring lumen and any relevant calibration information.

The relationship between the purging pressure and the pressure in the pressure monitoring lumen may be used to estimate the trachea pressure. For example, in certain embodiments, a trachea pressure value may be determined by the relationship:

P_{TRACHEA}=P_{LUMEN}−P_{PURGING}

where the trachea pressure is the pressure in the pressure monitoring lumen 14 after the purging pressure has been subtracted. For example, the above relationship may apply during a state when the purging pressure is greater than that of the trachea. If the trachea pressure is greater than that of the purging pressure, then the purging pressure and the tracheal pressure will reach equilibrium within lumen 14. In addition, when the purging pressure is greater than that of the trachea, the pressure transducer 44 may experience a certain delay in measuring deviations from purging pressure until the trachea pressure is greater than that of the purging pressure. This effect may be overcome by delaying or eliminating the pressure display (information read by the user) during certain portions of the respiratory cycle. For example, the display may indicate an error or zero when the monitor 50 determines that the measured pressure correlates to the purging pressure. In other embodiments, the monitor 50 may calibrate the trachea pressure when the purging pressure is greater than the trachea pressure based on a previously determined relationship (e.g. an empirically determined relationship that may be encoded on a calibration element). After the pressure transducer 44 measures a pressure greater than that of the flushing pressure, an algorithm could be used to extrapolate the data and create a wave form that illustrates the absolute pressure in the lung relative to the atmospheric pressure. In other embodiments, only peak trachea pressure may be displayed.

In one embodiment, the purging pressure may be sufficiently low and constant so that the effect on the trachea pressure is within an acceptable error, such as within 5%. In other embodiments, the purging pressure may be subtracted out by the monitor 50 to determine the trachea pressure. Depending on the level of purging pressure, the effect on the trachea pressure may be more pronounced at different points along the breathing cycle. As shown in FIG. 5, at exhalation, the purging pressure 100 may be greater than the minimum pressure 96, while during inhalation, the purging pressure 100 may be less than the peak trachea pressure 98 delivered by the tube 12. In such embodiments, although the purging flow is provided at a constant pressure, the ability of the pressure monitoring lumen 14 to be purged may depend on the whether the purging pressure 100 is greater than the trachea pressure. When the purging pressure 100 is greater than the trachea pressure, the gases in the pressure monitoring lumen 14 flow outward into the trachea, applying pressure to any secretions or buildup at the opening 20 or applying sufficient pressure to discourage such buildup. As such, it is contemplated that the purging pressure is generally established at a level greater than the pressure in the lungs for at least one point in the breathing cycle.

Monitor 50 may use the estimated trachea pressure to determine whether the breathing system 10 is achieving compliance. In certain embodiments, the estimated trachea pressure may be used to correct or adjust settings on a ventilator 22. For example, compliance may be associated with achieving target pressures in the airway during ventilation. If the target pressures in the airway are not achieved, the ventilator settings may be adjusted to increase or decrease the inspiratory pressure. Further, the estimated trachea pressure may be used to determine whether there is a blockage along the tube 12 by calculating the tube resistance using the pressure measurements and flow measurements taken at points closer to the ventilator 48, where a resistance increase may be indicative of a blockage or change in diameter of the tube 12. The monitor 50 may be configured to provide a graphical, visual, or audio representation of the estimated lung pressure. For example, ventilation compliance may be indicated by a green light indicated on a display, while a drop in pressure indicating a blockage in the tube 12 may trigger an alarm, which may include one or more of an audio or visual alarm indication. In one embodiment, the alarm may be triggered if the change in pressure is substantially greater than a predetermined value, substantially less than a predetermined value, or outside of a predetermined range.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of tracheal tube pressure, but these techniques may also be utilized for the measurement and/or analysis of the cuff pressure for any medical device inserted into a patient's airway. Rather, the various embodiments may cover all modifications, equivalents, and alternatives falling within the spirit and scope of the disclosure as defined by the following appended claims.

What is claimed is:

1. A system for trachea pressure measurement comprising:
a tracheal tube comprising a conduit configured to be inserted into the trachea of a subject to deliver respiratory gases to the subject;
a pressure monitoring lumen associated with the tracheal tube and in fluid communication with a pressure transducer;
a conduit configured to divert a portion of the respiratory gases to the pressure monitoring lumen;
a fluid reservoir in fluid communication with the conduit and configured to allow respiratory gases to accumulate to a first pressure within the reservoir; and
a pressure regulator configured to allow respiratory gases from the fluid reservoir to flow into the pressure monitoring lumen.

2. The system of claim 1, wherein the tracheal tube comprises an information element comprising a memory circuit storing calibration data for the system.

3. The system of claim 1, comprising a processor coupled to the pressure transducer, wherein the processor comprises encoded instructions for determining pressure based on measurements from the pressure transducer.

4. The system of claim 3, wherein the encoded instructions for determining pressure comprise instructions for accounting for flow from the fluid reservoir.

5. The system of claim 4, wherein the instructions for accounting for flow from the fluid reservoir comprise calibrating to a baseline pressure.

6. The system of claim 3, wherein the pressure regulator is coupled to the processor and the processor is configured to control the flow of respiratory gases from the fluid reservoir.

7. The system of claim 1, comprising one or more valves configured to facilitate one-way flow of respiratory gases into the fluid reservoir.

8. The system of claim 1, comprising one or more valves configured to facilitate one-way flow of respiratory gases from the fluid reservoir into the pressure monitoring lumen.

9. A method for determining trachea pressure in a patient under mechanical ventilation comprising:
communicating a trachea pressure present in a patient's trachea with a pressure monitoring lumen associated with a tracheal tube;
measuring the trachea pressure with a pressure transducer in fluid communication with the pressure monitoring lumen; and
diverting a portion of a patient's respiratory gases from a point on a respiratory circuit upstream of the patient's airway such that a generally continuous pressure of the respiratory gases is applied to the pressure monitoring lumen.

10. The method of claim 9, wherein the continuous pressure is less than about 5 cm H₂O.

11. The method of claim 9, wherein the continuous pressure is between about 1 cm H₂O and 5 cm H₂O.

12. The method of claim 9, wherein the continuous pressure is between about 2 cm H₂O and 3 cm H₂O.

13. The method of claim 9, wherein the continuous pressure is greater than a lowest pressure after exhalation and less than about 10 cm H₂O.

14. The method of claim 9, comprising determining a baseline pressure in the pressure monitoring lumen using the continuous pressure, and using the baseline pressure to determine a corrected tracheal pressure.

15. The method of claim 9, wherein diverting the respiratory gases comprises diverting gases at a point on the respiratory circuit downstream of a Y-connector.

16. The method of claim 9, comprising controlling the continuous pressure with a pressure regulator.

17. The method of claim 9, wherein diverting the respiratory gases comprises diverting gases to a fluid reservoir.

18. A method for determining trachea pressure comprising:
controlling a pressure regulator configured to allow respiratory gases accumulated within a fluid reservoir to flow into a pressure monitoring lumen associated with a tracheal tube, wherein the respiratory gases flow into the pressure monitoring lumen at a generally continuous pressure;
receiving information from a pressure transducer in fluid communication with the pressure monitoring lumen; and
determining a trachea pressure based at least in part on the information from the pressure transducer.

19. The method of claim 18, comprising determining the trachea pressure based on information relating to the continuous pressure.

20. The method of claim 18, comprising providing an indication to a caregiver based on the trachea pressure.
